(19) United States
(12) Patent Application Publication
(10) Pub. No.: US 2026/0265790 A1
(43) Pub. Date: Sep. 10, 2026

(54) CONSOLIDATED BIOPROCESSING OF COMPLEX CARBOHYDRATES TO D-ERYTHRO-ISOCITRIC ACID USING A FUNGUS

(71) Applicant: LEIBNIZ-INSTITUT FÜR NATURSTOFF-FORSCHUNG UND INFEKTIONSBIOLOGIE – HANSKNÖLL- INSTITUT –, Jena (DE)

(72) Inventors: Ivan Schlembach, Jena (DE); Miriam Agler-Rosenbaum, Jena (DE); Lars Regestein, Jena (DE); Gerhard Kerns, Leipzig (DE)

(21) Appl. No.: 19/165,936

(22) PCT Filed: Mar. 7, 2024
(86) PCT No.: PCT/EP2024/056067

§ 371 Date: Sep. 16, 2025

(30) Foreign Application Priority Data
Mar. 28, 2023 (DE) 10 2023 107 876.3

(51) Int. Cl. C12P 7/48; C12N 1/145; C12R 1/66; C12R 1/80

(52) U.S. Cl. CPC C12P 7/48; C12N 1/145; C12P 2203/00; C12R 2001/66; C12R 2001/80

(57) ABSTRACT

The present invention relates to a method for the one-step bioproduction of isocitric acid from a cellulose substrate in a suitable fungus of the genus Penicillium, Talaromyces or Aspergillus. The problem of the present invention is solved by providing a method for the one-step bioproduction of isocitric acid from a complex carbohydrate substrate in a suitable fungus, in particular of the genus Penicillium, Talaromyces or Aspergillus, the method comprising, cultivating said fungus, in particular of the genus Penicillium, Talaromyces or Aspergillus, in a culturing vessel in a suitable medium comprising complex carbohydrate at a temperature of between 25° and 40° C., under nitrogen limitation, and at a pH of the culture at between 4 and 7, preferably between 4.8 to 5.8, whereby isocitric acid is produced.

The present invention relates to a method for the one-step bioproduction of isocitric acid (ICA) from a complex carbohydrate substrate, such as cellulose, using a suitable fungus, in particular of the genus Penicillium, Talaromyces or Aspergillus.

### BACKGROUND OF THE INVENTION

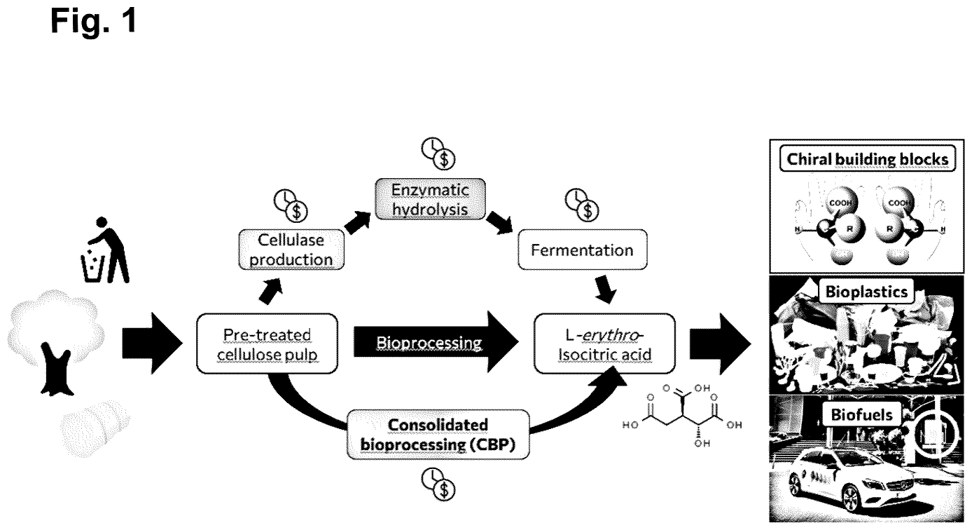

Consolidated bioprocessing (CBP) is an emerging field of research focusing on the direct conversion of cheap cellulosic waste streams into valuable bulk and specialty chemicals including biofuels (See FIG. 1). Thereby, costly separate enzyme production and cellulose hydrolysis can be skipped and performed in one process step in a single bioreactor. This offers great potential to improve the efficiency and economics of cellulose valorization, which still imposes a major barrier for large scale commercialization of these processes. However, such process presupposes a biocatalyst that is able to simultaneously degrade complex lignocellulose while being capable to produce a target product of interest. Yet, natural organisms that possess both of these desirable traits are scarce [1].

Only few anaerobic bacteria are known to convert cellulose to valuable products, including Thermoanaerobacterium thermosaccharolyticum for butanol, Clostridium phytofermentans for ethanol production or Fibrobacter succinogenes for succinate production [2-5]. Therefore, researchers have tried to either genetically engineer native cellulolytic organisms to enable the biosynthesis of a target product (native cellulolytic strategy) or to recombinantly express cellulase enzymes in organisms, which are natural producers of the target product (recombinant cellulolytic strategy). Both of these approaches result in the generation of genetically modified organisms (GMOs). Another approach is the application of a defined co-culture of a specialized cellulase producer with a desired target production strain, which has been proven successful for a variety of products including itaconic, fumaric, butyric and lactic acid as well as ethanol, isobutanol, butanol and acetone [6-12].

The conventional route for lignocellulose valorization starts with a separate production of cellulase enzymes using a suitable host. The produced enzymes are then added to a hydrolysis tank, where the pretreated cellulose substrate is converted under optimized conditions into a sugar syrup containing sugar mono- and oligomers. The syrup is finally fed to a fermentation tank, where the sugars are fermented into the product of interest. The three separate processes require three separate bioreactors and peripheral equipment, which typically leads to increased capital and operating costs as well as decreased space-time yield compared to a consolidated bioprocess (CBP) [13]. In a consolidated bioprocess, the pretreated cellulose substrate is directly converted into the target product within a single bioreactor. The cellulase enzymes are produced directly within the process and the cellulose hydrolysis takes places simultaneously to the fermentation of the released sugars into the target product, which relieves product inhibition of cellulases and thereby boosts hydrolysis efficiency. In addition to techno-economic benefits, such process results in considerable simplification of cellulose conversion processes and ease of operation.

Among these different CBP approaches, the only strategies that reached relevant product quantities are co-cultures as well as native cellulolytic approaches with GMOs. However, genetically engineered organisms are preferentially avoided in large scale bioprocesses for biocontainment and complex licensing reasons and co-cultures are difficult to control [14]. This hinders the application of these CBPs in large scale industrial processes.

The only current industrial processes that use second generation lignocellulosic substrates (e.g. the Clariant Sunliquid® process) still rely on the classical sequential separated process steps based on axenic cultures because currently, none of the CBP approaches are suitable alternatives to the conventional processes with respect to final product concentration, volumetric productivities and simplicity of the process [15, 16].

Kamzolova S V, et al. (in: Large-Scale Production of Isocitric Acid Using Yarrowia lipolytica Yeast with Further Down-Stream Purification. BioTech. 2023; 12(1):22. https://doi.org/10.3390/biotech12010022) disclose a process of D-threo-isocitric acid production from rapeseed oil using yeast Yarrowia lipolytica VKM Y-2373 in a 500-L fermenter. The producer synthesized 64.1 g/L D-threo-isocitric acid with a product yield of 0.72 g/g and a productivity 0.54 g/L-h. They also developed a purification method, including a cell separation, clarification, concentration, acidification, and crystallization process, which resulted in the formation of the crystals of monopotassium salt of D-threo-isocitric acid with a purity of 99.0-99.9%.

CN106148209B discloses a genetically modified strain of the cellulolytic fungus Myceliophthora thermophila that is able to convert cellulose directly into malic acid and succinic acid. Additional acids as mentioned are fumaric acid, oxaloacetic acid, glutaric acid and adipic acid. Concentrations of 180 g/L malic acid and 20 g/L succinic acid were achieved in a fed-batch bioprocess with cellulose, with a volumetric productivity of 1 g/L/h.

The cellulolytic properties of P. verruculosum are well known, and it was shown that P. verruculosum cellulases are more efficient than established cellulase-cocktails that were obtained with T. reesei. DE102011014444A1 discloses strains of P. verruculosum showing improved cellulase production, which were used for obtaining bioethanol from lignocellulose.

Schlembach I et al. (in: Consolidated bioprocessing of cellulose to itaconic acid by a co-culture of Trichoderma reesei and Ustilago maydis. Biotechnol Biofuels. 2020 Dec. 14; 13(1):207. doi: 10.1186/s13068-020-01835-4. PMID: 33317635; PMCID: PMC7737373) describe a fully consolidated bioprocess (CBP), which is capable of directly converting recalcitrant cellulose into itaconic acid without the need for separate cellulose hydrolysis including the application of commercial cellulases. The process is based on a synthetic microbial consortium of the cellulase producer Trichoderma reesei and the itaconic acid producing yeast Ustilago maydis, i.e. the application of a defined co-culture of a specialized cellulase producer with a desired target production strain. This process requires an efficient co-culture of two different microorganisms, and only discloses a theoretical and complex conversion of cellulose into itaconic acid.

In the context of the fossil resources transition to a circular economy, urgently new methods for a sustainable production of chemical building blocks as well as carriers of energy are needed.

It is therefore an object of the present invention to provide a biocatalytic consolidated process in order to provide the efficient conversion of complex sugar substrates, such as cellulose, into more valuable products in general, without the use of GMOs (regulatory advantages), and a production of enantiomer-pure D-erythro-isocitric acid as a platform and fine chemical. A further object of the present invention is the development of a natural, non-GMO that is capable to convert recalcitrant cellulose directly and at high productivity into enantiopure D-erythro-isocitric acid. Other objects and advantages will readily become apparent for the person of skill from studying the following more detailed description and examples.

The problem of the present invention is solved by providing a method for the one-step bioproduction of isocitric acid from a complex carbohydrate substrate in a suitable fungus, in particular of the genus Penicillium, Talaromyces or Aspergillus, the method comprising, cultivating said fungus, in particular of the genus Penicillium, Talaromyces or Aspergillus, in a culturing vessel in a suitable medium comprising complex carbohydrate at a temperature of between 25° and 40° C., under nitrogen limitation, and at a pH of the culture at between 4 and 7, preferably between 4.8 to 5.8, whereby isocitric acid is produced. Preferred is the method according to the present invention, wherein the isocitric acid is D-erythro-isocitric acid ((2S, 3S)-isocitric acid). Further preferred is the method according to the present invention, wherein the Penicillium is selected from a non-GMO and/or native or wildtype cellulolytic strain of Penicillium, in particular Penicillium verruculosum, such as Penicillium verruculosum M28-10 or Penicillium verruculosum M28-9.

In a preferred embodiment of the method according to the present invention, no nitrogen is added to the culture, or the nitrogen is limited to less than 0.5 g/L of the culture. The nitrogen source may be (NH₄)₂SO₄ or any other source of nitrogen.

In the context of the present invention, the term “nitrogen limitation” shall mean a state of the fungal culture method according to the present invention, where the cell biomass production, and preferably the cellulase production, is dependent from, at least substantially dependent from, the availability of nitrogen in the culture. How to achieve and control nitrogen limitation in a cell culture is known to the person of skill.

In a preferred embodiment of the method according to the present invention, the method further comprises the step of isolating said isocitric acid as produced from said culture, comprising, for example in-situ removal of the isocitric acid.

In a preferred embodiment of the method according to the present invention, about 30-45 g/L, preferably about 40 g/L isocitric acids are produced, comprising more than 90%, preferably more than 95%, and more preferably more than 99%, such as about 99.5% D-erythro-isocitric acid.

The problem of the present invention is solved by providing a kit, comprising materials for performing a method according to the present invention, such as, for example, a culture of a suitable fungus, in particular of the genus Penicillium, Talaromyces or Aspergillus, and a suitable culture medium comprising lacking nitrogen and having a pH at between 4 to 7, preferably between 4.8 to 5.8, more preferably at 5.5.

The problem of the present invention is solved by providing the use of the kit according to the present invention for the one-step bioproduction of isocitric acid from a cellulose substrate in a suitable fungus, in particular of the genus Penicillium, Talaromyces or Aspergillus.

Research has been carried out for some time on the utilization of renewable lignocellulose, such as for the production of alcohols and organic acids, which serve as platform chemicals for numerous products. Classically, the biotechnological utilization of cellulose requires three separate individual processes: the production of cellulolytic enzymes, the saccharification of cellulose by means of these enzymes and finally the biocatalytic conversion of the sugars into the target molecule. Each of the three individual processes is divided into further process steps, so that a multi-stage process chain is created, with low efficiency due to its length and complexity. Above all, the first step, enzyme production, represents a considerable cost factor. The reduction of this entire process chain into a single reaction step (=process intensification or consolidation) therefore holds enormous potential to make cellulose valorization much more efficient and thus economical.

In the context of the present invention, the term “cellulase” or “cellulases” shall relate to any of several enzymes produced by fungi that catalyze hydrolysis of various lignocellulosic materials, i.e., the decomposition of complex carbohydrates like cellulose, hemicellulose, starch, pectin, and related polysaccharides. The term includes any naturally occurring mixture or complex of various such enzymes, that act serially or synergistically to decompose lignocellulosic material.

In the context of the present invention, the term “complex carbohydrate” shall refer to a compound that is composed of three or more monosaccharide or uronic acid units bound together in a branched or unbranched chain. Preferred examples are lignocellulose, cellulose, starch, pectin, and related polysaccharides.

Antonov E, et al. (in: J. Process relevant screening of cellulolytic organisms for consolidated bioprocessing. Biotechnol Biofuels. 2017 Apr. 24; 10:106. doi: 10.1186/s13068-017-0790-4. PMID: 28450887; PMCID: PMC5402656) disclose a novel method for evaluating the in situ cellulose consumption rate of different cellulase producers. Various cellulase producers were analyzed by quantifying initial cellulase activity under target process conditions. Promising candidates were then characterized online by monitoring their respiration activity metabolizing cellulose to assess the growth and enzyme production dynamics. The screening of five different cellulase producers with the freeze assay identified Trichoderma reesei and Penicillium verruculosum as most promising. The measurement of the respiration activity revealed a retarded induction of cellulase production for P. verruculosum but a similar cellulase production rate afterwards, compared to T. reesei. The freeze assay measurement depicted that P. verruculosum reaches the highest initial carbon release rate among all investigated cellulase producers. After a modification of the cultivation procedure, these results were confirmed by the respiration activity measurement. T. reesei and P. verruculosum were identified as compatible candidates for the chosen model process.

In the present context the term “one-step bioproduction” shall mean to relate to a process that reduces the entire process chain into a single reaction step (=process intensification or consolidation), and therefore holds enormous potential to make complex carbohydrate, such as cellulose, recycling much more efficient and thus economical. As mentioned below, a preferred method is a fed-batch method in a culture vessel.

Under conditions optimized for cellulase production, the inventors previously demonstrated using respirometry that P. verruculosum can reach in-situ cellulose metabolization rates of 0.9 g/L/h at low cellulose concentration (30 g/L) and even 1.7 g/L/h at high cellulose concentrations (120 g/L) [19]. For erythro-isocitric acid formation, glucose consumption rates of 1.4 to 2.6 were measured depending on the starting concentration of nitrogen, which resulted in isocitric acid production rates between 0.7 to 1.2 g/L/h, respectively. However, the high initial glucose consumption rates observed with high nitrogen supplementation could be only sustained for limited time due to the higher biomass concentration, affecting the viscosity of the broth. Quickly the broth became too viscous for efficient mixing and aeration, limiting the metabolism. Therefore, the method was not suitable for an industrial process approach.

As mentioned above, in a first aspect of the present invention, the object of the present invention is solved by a method for the one-step bioproduction of isocitric acid from a complex carbohydrate substrate in a suitable fungus, in particular of the genus Penicillium, Talaromyces or Aspergillus, the method comprising, cultivating said fungus, in particular of the genus Penicillium, Talaromyces or Aspergillus, in a culturing vessel in a suitable medium comprising the complex carbohydrate at a temperature of between 25° and 40° C., under nitrogen limitation, and at a pH of the culture at between 4 to 7, preferably between 4.8 to 5.8, whereby isocitric acid is produced.

Initially, it was surprisingly found that isocitric acid can be produce using the inventive method in an amount of more than about 30% when compared to a bioproduction of isocitric acid from glucose as a substrate. An example of such a comparison is described herein and shown, for example, in table 2. A possible explanation for the different maximum concentration could be a result of relieved weak acid toxicity due to in-situ precipitation or complexation of Calcium-isocitrate with CaCO₃. The present method therefore has big potential for improvement since up to 100 g/L erythro-isocitric acid have been achieved in glucose-based cultivations using CaCO₃. It is anticipated by the inventors that the present method allows for higher amounts of isocitric acid production per supplied complex carbohydrate unit, i.e., equal to the glucose-based process, if a diffusion of the weak acid as produced through the membrane into the fungal cell is avoided. This may be achieved by a control of the pH using, e.g., suitable titration, and/or by continuous removal of the acid using in-situ removal (see below). A promising strategy to further increase the titer of isocitric acid in CBP, would be the use of CaCO₃ or CaOH to control the pH instead of NaOH, as it will precipitate isocitrate in situ, thereby relieving weak acid toxicity and increasing final production titers. Further optimization is possible by tuning the tradeoff regarding erythro-isocitric acid yield and cellulase activity in regard to pH and temperature.

Preferred is the method according to the present invention, wherein the isocitric acid that is produced is D-erythro-isocitric acid.

Isocitric acid has a wide range of applications, but these have not yet been realized due to the extremely high price of the compound. This is because commercial isocitric acid has so far only been extracted from certain plants of the Crassulaceae family, with very low efficiency (250 kg of plant material for 16 g of isocitric acid) [8].

In addition, the separation of isocitric acid and citric acid, which is often a by-product, is relatively complex. Full chemical synthesis is also complex and inefficient. Recently, a biotechnological process has been developed for the production of enantiomerically pure D-threo-isocitric acid using the yeast Yarrowia lipolytica starting from sunflower and rapeseed oil [9]. Using wild-type strains of Y. lipolytica, yields of 0.8 g/g and titers between 70 and 110 g/L of isocitric acid have been obtained from ethanol or rapeseed oil as a substrate [10, 11]. GMO strains produce up to 137 g/L D-threo-isocitric acid from glucose [12]. In the process with Y. lipolytica, however, citric acid (about 10 g/L) is always produced as a by-product, which must be separated from the isocitric acid.

The company Chiroblock now sells a D-threo-isocitric acid produced with Y. lipolytica with an enantiomer purity of >98% for 19,000 €/kg [13]. The company has also published an interesting white paper with many potential applications for isocitric acid [8]. Rejuvenating and antioxidant effects have been observed, which allow an application in cosmetics and dietary supplements. However, the biological effects of enantiomerically pure D-erythro-isocitric acid are still unclear.

TABLE 1
Overview isocitric acid production (ICA: isocitric acid, CA: citric acid)

| | | Price for | | | | | |
|---|---|---|---|---|---|---|---|
| | | substrate | | | | | |
| | | per ton | Yield | | Productivity | | |
| | | [€] (pro | [g/g] | Titer | [g/L/h] | Ratio | |
| Organism | Substrate | kg Isocit) | (max) | [g/L] | (max) | ICA/CA | Ref |
| Y. lipolytica | Glucose | 500 | 0.74 | 137 | 1.32 | 7.4:1 | [12] |
| | | (0.68) | | | | | |
| Y. lipolytica | Rapeseed | 1160 | 0.82 | 70.6 | 0.8 | 3.2:1 | [10] |
| | oil | (1.41) | | | | | |
| Y. lipolytica | Ethanol | 680 | 0.8 | 109.6 | 1.35 | 4:1 | [11, 14] |
| | | (0.85) | | | | | |
| P. | Glucose | 500 | 0.85 | 84.8 | 0.44 | n.d. | [15] |
| purpurogenum | | (0.59) | | | | | |
| P. | Cellulose | * 130 | 0.24 | 38.4 | 0.7 | >100:1 | This |
| verruculosum | | (0.45) | (0.29) | | | | invention |
| P. | Glucose | 500 | 0.6 | 101 | 0.7 (1.2) | >100:1 | This |
| verruculosum | | (0.66) | (0.76) | | | | invention |
| | | | | | | | (comp.) |

* The current cost of wheat straw is €85/ton [19]. With an average carbohydrate content of 65%, this results in a substrate price of approx. 130€/ton of glucose equivalents from wheat straw hydrolysate (without enzyme costs).

The only documented microbial production of the sister enantiomer D-erythro-isocitric acid dates from 1958-1961 by researchers Sakaguchi Kinichiro and Beppu Teruhiko.

U.S. Pat. No. 2,949,404A discloses a process for the production of D-erythro-isocitric acid with various Penicillium strains, including P. verruculosum (U.S. Pat. No. 2,949,404A, CA612398A, GB852486A: Method of producing allo-isocitric acid by fermentation). D-erythro-isocitric acid (formerly named Allo-isocitric acid) is produced by a fermentation process in which a fungus is cultivated in a liquid or solid nutrient medium comprising various carbohydrate materials, inorganic salts and nitrogen sources, and allo-isocitric acid is recovered from the fermented medium. The fungi which may be used belong to the genus Penicillium. As specific substrates, glucose, saccharose, fructose, mannose, xylose, arabinose and inulin are mentioned. Complex substrates, like cellulose are not mentioned.

Here, the inventors demonstrate for the first time a natural, non-GMO that is capable to convert recalcitrant cellulose directly and at high productivity into enantiopure D-erythro-isocitric acid. This organism is the fungus Penicillium verruculosum, which was first isolated by B. Peyronel in 1913 and later bred into an excellent cellulase producer via a classical mutagenesis approach [17, 18]. The inventors previously, as an example, demonstrated the potential of P. verruculosum as a superior cellulase producer for co-culture based CBP applications at low pH in a process relevant screening [19]. Thereby, P. verruculosum could even outperform the well-established cellulase producer T. reesei RUT-C30. However, unaware of its erythro-isocitric acid production capabilities, the inventors never exploited P. verruculosum as CBP organism in axenic culture at that time.

D-Erythro-isocitric acid is a rare enantiomer of D-threo-isocitric acid, which is the ubiquitous intermediate from the TCA cycle. The microbial production of erythro-isocitric acid has up to now only been discovered by a single research group in 1957 [20]. Beppu and colleagues first discovered the production of this unusual isocitric acid enantiomer in a strain of Penicillium purpurogenum Stoll var. rubri-sclerotium Thom. No. 1148. At this time, the acid was called allo-isocitric acid.

D-Erythro-isocitric acid has a promising potential as novel platform molecule for both bulk and fine chemistry. It was initially patented by Sakaguchi and Beppu et al. as an acidulant for beverages but has never been commercially exploited [21]. The current market price of racemic mixture of isocitric acids is about 18000 $/kg, which strongly limits its large-scale application. Still, if it could be produced at competitive price, isocitric acid has many application opportunities. It could serve as a superior anticoagulant for blood preservation and could completely substitute conventional citrate, EDTA and heparin buffers [22, 23]. Many positive anti-aging properties have been reported for isocitric acid, which opens application possibilities for food and cosmetics applications [23]. The fact that the acid can be produced with high enantioselectivity makes it an interesting candidate as building block for chiral synthesis. The sister molecule D-threo-isocitric acid is already marketed for this purpose by the company “Chiroblock” and is used for the synthesis of the HIV medication Darunavir [24, 25]. Furthermore, isocitric acid can be easily converted into itaconic acid, which belongs to the top value-added platform chemicals from biomass and has a huge market potential for substituting acrylate monomers [26, 27].

The inventors demonstrate the potential of P. verruculosum for the direct conversion of complex carbohydrates, such as cellulose, to D-erythro-isocitric acid. By comparing the different process requirements of the different subprocesses cellulase production, complex carbohydrate hydrolysis and isocitric acid production, the inventors show a possible reason why this natural, process ready CBP organism might has been overlooked for decades.

Further preferred is the method according to the present invention, wherein the Penicillium is selected from any suitable and preferably a non-GMO and/or native or wildtype cellulolytic strain of Penicillium, in particular Penicillium verruculosum, such as Penicillium verruculosum M28-10, or Penicillium verruculosum M28-9. The production in other Penicillium strains may include Penicillium sclerotiorum, Penicillium expansum, and Penicillium aculeatum. Also suitable may be Penicillium purpurogenum Stoll var. rubri-sclerotium Thom (sclerotia producing strains and non-sclerotigenic type), Penicillium sclerofiorum van Beyma, Penicillium expansum (Link) Thom, Penicillium verruculosum Peyronel and Penicillium aculeatum Raper and Fennell. These strains are publicly available, such as, for example Penicillium verruculosum-M28-10b as DSM 24317.

In the context of the present invention, the temperature at which the method is performed has to reflect a balance between growth optimum of the fungus and the respective enzymatic activities of enzymes involved, as well as the temperature as required for an efficient conversion into the desired isocitric acid as produced from said culture. Preferred is a method according to the present invention, wherein the temperature for the culture is held at between about between 30° C. and 37° C., most preferred at about 37° C.

Another important parameter of the method is the pH of the culture. While a high pH in combination with nitrogen limitation is a requirement for efficient isocitric acid formation, cellulase activity and thus complex carbohydrate, e.g., cellulose, conversion will be negligible and no cellulases can be produced under these conditions. In contrast, a low pH will ensure high cellulase activity and thus complex carbohydrate, e.g., cellulose, conversion efficiency but prevent isocitric acid formation regardless of nitrogen availability. Preferred is a method according to the present invention, wherein the pH of the culture is at about 5.3 to 5.7, preferably the pH is regulated to about 5.5. Usually, 10% NaOH is used for regulating the pH. The pH can also be regulated by CaCO₃, CaOH or other base addition, which could be even beneficial due to Ca-isocitrate precipitation and complexation.

Another important aspect of the method is the source of the complex carbohydrate, in particular cellulose, as this has a strong influence on the economic efficiency of the method. Preferably, the complex carbohydrate substrate, such as cellulose, is derived from agricultural or forestry residues such as straw or energy crops that grow on non-arable land. The straw can be obtained from any suitable grain, like wheat, rice, barley, and even from sugar cane. Other sources of complex carbohydrates are potato peels, beet pulp, microalgae, macroalgae and the like.

The present culture is preferably performed with air- or O₂-gassing and stirring. The method according to the present invention includes any suitable stirring and introduction of gasses that promote the growth and conversion. The stirring rate is adjusted in order to provide a sufficient level of dissolved oxygen, a sufficiently homogeneous distribution of the cellulosic material in the culture, and to further control the viscosity of the culture, while avoiding damaging the cells in the culture through applying excessive shear forces. Optimal stirring is controlled through an average volumetric power input of between 0.5 to 10 kW m³ of culture.

Another important aspect of the method is nitrogen limitation in the culture (see also above). Preferred is a method according to the present invention, wherein no nitrogen is added to the culture, as this is introduced by the substrate (e.g. straw) as used. The nitrogen limitation can be limited at any suitable time during the culture. While a high pH in combination with nitrogen limitation is a requirement for efficient isocitric acid formation, cellulase activity and thus cellulose conversion will be negligible and no cellulases can be produced under nitrogen limitation conditions. Therefore, the cellulase production has to occur in the initial cultivation phase, when nitrogen is still available while the isocitric acid formation will start in sequence to cellulase formation, once nitrogen has become the growth limiting nutrient.

As mentioned above, a main advantage of the inventive method is the simplicity in that a one-step bioproduction is achieved with a non-GMO in a process that reduces the entire process chain into a single reaction step (=process intensification or consolidation), and therefore holds enormous potential to make cellulose recycling and valorization much more efficient and thus economical. Any suitable single-step procedures may be used in the context of the present invention, in a preferred embodiment according to the present invention, the culture is performed is a fed-batch culture, for example comprising feeding of the complex carbohydrate in order to maintain a suitable level of substrate in the culture, for example at above about 60 g/L.

In another aspect of the present invention, the method according to the present invention further comprises the step of isolating said isocitric acid as produced from said culture. This may be done in a conventional way, such as filtering the resulting culture broth, adding methyl alcohol or acetone to the filtered culture broth, whereby D-erythro-isocitric acid salt precipitates, and recovering the precipitated salt. Other methods include reactive extraction, calcium precipitation, ion exchange, electrodialysis, etc., and the person of skill is well aware of these. For in-situ removal, for example a heat-based calcium carbonate precipitation method in a bypass can be used, since the solubility of calcium isocitrate is decreasing with increasing temperature (Hong Cheng, André C. Garcia, Ning Tang, Bente P. Danielsen, Leif H. Skibsted, Combinations of isocitrate and citrate enhance calcium salt solubility and supersaturation robustness, International Dairy Journal, Volume 85, 2018, Pages 225-236, ISSN 0958-6946, https://doi.org/10.1016/j.idairyj.2018.06.009).

As mentioned above, another main advantage of the inventive method is the effective production of the valuable isocitric acids at rates comparable to the tedious and expensive production using individual sugars, such as glucose. At least about 30-45 g/L, preferably about 38.4 or 40 g/L isocitric acids are produced, comprising more than 90%, preferably more than 95%, and more preferably more than 99%, such as about 99.5% D-erythro-isocitric acid. This means that basically the desired product can be directly obtained.

In the present context the term “about” shall indicate a deviation of +/−10% from the value as given, if not indicated otherwise.

Another aspect of the present invention then relates to a kit, comprising materials for performing a method according to the present invention, such as, for example, a culture of a suitable fungus, in particular of the genus Penicillium, Talaromyces or Aspergillus, and a suitable culture medium comprising lacking nitrogen and having a pH at between 4 to 7, preferably between 4.8 to 5.8, more preferably at 5.5. Other possible materials include a manual, and the cellulose source(s).

Another aspect of the present invention then relates to the use of the kit according to the present invention for the one-step bioproduction of isocitric acid from a cellulose substrate in a suitable fungus, in particular of the genus Penicillium, Talaromyces or Aspergillus, preferably according to a method according to the present invention.

Here, the inventors demonstrate for the first time, a process-ready, non-GMO wildtype organism, capable of efficient CBP of cellulose to D-erythro-isocitric acid. It was demonstrated that cellulose can be directly converted to D-erythro-isocitric acid at a rate similar to the glucose-based process. The example shows that even with the limited amount of nitrogen supplied, a complete CBP process with in-situ cellulase production is possible and reaches sufficient cellulase activity to compete with SHF of SSF processes. This shows that lignocellulose fermentation can be straight forward and as convenient as glucose fermentation. Considering its remarkable CBP performance, P. verruculosum will serve as one promising chassis for the production of other bio-commodities in the future.

The presented invention shows that natural occurring organisms are well capable of fast and efficient conversion of cellulose into valuable molecules but only under very defined conditions. Even using the performance data already achieved in this invention the process has high economic potential. With a current selling price of 18000 $/kg for a racemic mixture of isocitric acid, the original glucose-based process initially disclosed by Beppu et al. would be already profitable in the high value fine chemistry market. Considering that the achieved yields, productivities and even the purification process via Ca-isocitate precipitation are similar to citric acid production, it can be predicted that enantiopure D-erythro-isocitric acid could be easily produced for a similar selling price of citric acid (2-4$/kg) based on glucose or molasse. However, the fact that the inventors demonstrated it can be directly produced from cellulose makes the process even more interesting. In comparison to glucose (400$/ton) the current price of wheat straw is (70-90€/ton). Having a carbohydrate content of ~65%, the price of fermentable wheat straw carbohydrate would be 110-140€/ton excluding the cost of enzyme for saccharification, cutting substrate related costs to approx. ⅓ of conventional substrates. Although the g/g yield in the cellulose-based process is still lower than using glucose, this is no big drawback in cellulose fermentation because remaining substrate after the fermentation can be easily burned for combined heat and power generation to make the process completely self-sufficient.

The invention therefore has the following advantages:
The invention allows the direct production of D-erythro-isocitric acid from cellulose.
The invention allows the efficient conversion of cellulose into a platform chemical with a wide range of uses without the use of genetically modified organisms.
Due to the use of cellulose as a raw material, the process is not only sustainable but also economically interesting due to the low price of e.g. agricultural residues such as straw. Assuming that all other process costs are comparable, the cellulose process already offers the most favorable substrate cost-to-product ratio (Table 1).
Even if there is not yet a large market for D-erythro-isocitric acid, the production is still interesting. Isocitric acid can easily be converted to itaconic acid, which has a very large market in the acrylate monomer segment.
The enantiomeric purity is >99.5%, which is higher than that of D-threo-isocitric acid from the biotechnological process with Y. lipolytica.
No by-products other than D-erythro-isocitric acid are formed, which simplifies purification.
The cellulose used as a substrate is not completely degraded and can be used directly as a filtration aid when processing the culture broth. The filter cake can then be incinerated and provide process heat and electricity, e.g. for pre-treating the lignocellulose. As a result, yield losses can be compensated. The remaining ash, in turn, after elemental analysis, can be used to formulate the mineral medium to enable a closed process with no waste.

The present invention relates to the following items:

Item 1: A method for the one-step bioproduction of isocitric acid from a complex carbohydrate substrate in a suitable fungus, in particular of the genus Penicillium, Talaromyces or Aspergillus, the method comprising, cultivating said suitable fungus, in particular of the genus Penicillium, Talaromyces or Aspergillus, in a culturing vessel in a suitable medium comprising the complex carbohydrate at a temperature of between 25° and 40° C., under nitrogen limitation, and at a pH of the culture at between 4 to 7, preferably between 4.8 to 5.8.

Item 2. The method according to Item 1, wherein the isocitric acid is D-erythro-isocitric acid ((2S,3S)-isocitric acid).

Item 3. The method according to Item 1 or 2, wherein the Penicillium is selected from a non-genetically modified and/or native or wildtype cellulolytic strain of Penicillium, in particular Penicillium verruculosum, such as Penicillium verruculosum M28-10 or Penicillium verruculosum M28-9.

Item 4. The method according to any one of Items 1 to 3, wherein the temperature is at about 30° C. to 37° C., preferably at about 37° C.

Item 5. The method according to any one of Items 1 to 4, wherein the pH of the culture is at between 5.3 to 5.7, preferably at about 5.5.

Item 6. The method according to any one of Items 1 to 5, wherein said complex carbohydrate substrate is selected from agricultural or agroforestry residues containing starch, pectin or cellulose, wherein for example said complex carbohydrate substrate is derived from straw.

Item 7. The method according to any one of Items 1 to 6, wherein culturing comprises stirring at a rate based on a power input of between 0.5 to 10 kw m³ of cell culture.

Item 8. The method according to any one of Items 1 to 7, wherein no nitrogen is added to the culture, or the nitrogen is limited to less than 0.5 g/L of the culture.

Item 9. The method according to any one of Items 1 to 8, wherein the gassing rate of the culture is at between 0.2 and 2 volumes of air sparged per unit volume of growth medium per minute (VVM), preferably at about 1 VVM.

Item 10. The method according to any one of Items 1 to 9, wherein said culture is performed is a fed-batch culture, for example comprising feeding of the complex carbohydrate in order to maintain the level of substrate in the culture at above about 60 g/L.

Item 11. The method according to any one of Items 1 to 10, further comprising the step of isolating said isocitric acid as produced from said culture, comprising, for example in-situ removal of the isocitric acid.

Item 12. The method according to any one of Items 1 to 11, wherein at least about 30-45 g/L, preferably about 38.4 or 40 g/L isocitric acids are produced, comprising more than 90%, preferably more than 95%, and more preferably more than 99%, such as about 99.5% D-erythro-isocitric acid.

Item 13. A kit, comprising materials for performing a method according to any one of Items 1 to 12, such as, for example, a culture of a suitable fungus, in particular of the genus Penicillium, Talaromyces or Aspergillus, and a suitable culture medium comprising lacking nitrogen and having a pH at between 4 to 7, preferably between 4.8 to 5.8, more preferably at 5.5.

Item 14. Use of the kit according to Item 13 for the one-step bioproduction of isocitric acid from a cellulose substrate in a suitable fungus, in particular of the genus Penicillium, Talaromyces or Aspergillus.

The invention will now be described further in the following examples with reference to the accompanying figures, nevertheless, without being limited thereto. For the purposes of the present invention, all references as cited are incorporated by reference in their entireties.

FIG. 1 shows a schematic overview of conventional versus consolidated bioprocessing of cellulose. The conventional route for lignocellulose valorization starts with a separate production of cellulase enzymes using a suitable host. The produced enzymes are then added to a hydrolysis tank, where the pretreated cellulose substrate is converted under optimized conditions into a sugar syrup containing sugar mono- and oligomers. The syrup is finally fed to a fermentation tank, where the sugars are fermented into the product of interest. The three separate processes require three separate bioreactors and peripheral equipment, which typically leads to increased capital and operating costs as well as decreased space-time yield compared to a consolidated bioprocess (CBP) [13]. In a consolidated bioprocess, the pretreated cellulose substrate is directly converted into the target product within a single bioreactor. The cellulase enzymes are produced directly within the process and the cellulose hydrolysis takes places simultaneously to the fermentation of the released sugars into the target product, which relieves product inhibition of cellulases and thereby boosts hydrolysis efficiency. In addition to techno-economic benefits, such process results in considerable simplification of cellulose conversion processes and ease of operation.

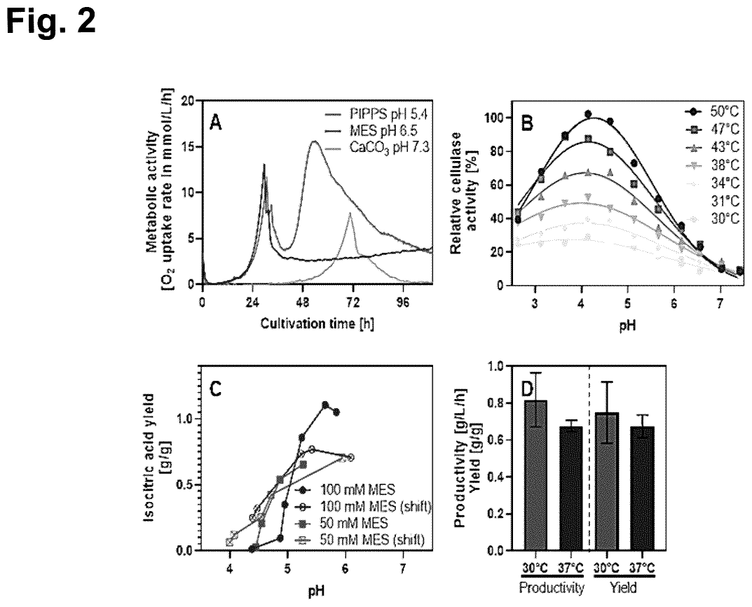

FIG. 2 shows the process optima of P. verruculosum for the different subprocesses leading from cellulose to D-erythro-isocitric acid. A) In situ cellulolytic activity of P. verruculosum at different pH values using different buffers (each at 100 mM concentration) as evaluated by online respirometry. Thereby the O₂ consumption rate after consumption of glucose, correlates to the cellulose consumption rate. The cultivations were performed in medium containing 5 g/L glucose, 2 g/L peptone and 30 g/L cellulose as carbon sources and 7.6 g/L (NH₄)₂SO₄ as nitrogen source. B) shows the activity profile of P. verruculosum cellulase enzymes. Cell free supernatant of P. verruculosum was buffered to various pH values with 75 mM phosphate-citrate buffer and the release of reducing sugars was quantified photometrically using DNS-reagent after 1 h incubation with a disc of filter paper at various temperatures. All values were normalized to the maximum recorded value. C) shows the yield of isocitric acid in g/g of consumed glucose measured from P. verruculosum cultivations performed in medium containing 1.25 g/L (NH₄)₂SO₄ as nitrogen source, 60 g/L glucose, buffered to pH 6.7 with either 100 mM (blue values) or 50 mM (red values) MES buffer. Isocitric acid production stopped below pH 4. To investigate if isocitric acid production restarts after pH readjustment, both cultures were shifted to pH 6.5 using 10M NaOH after the pH drop. Values recorded after the pH shift are shown with open crossed symbols. D) shows the yield and productivity measured at different cultivation temperatures for P. verruculosum cultivations containing 60 g/L glucose buffered with 40 g/L CaCO₃, error bars show 95% confidence intervals.

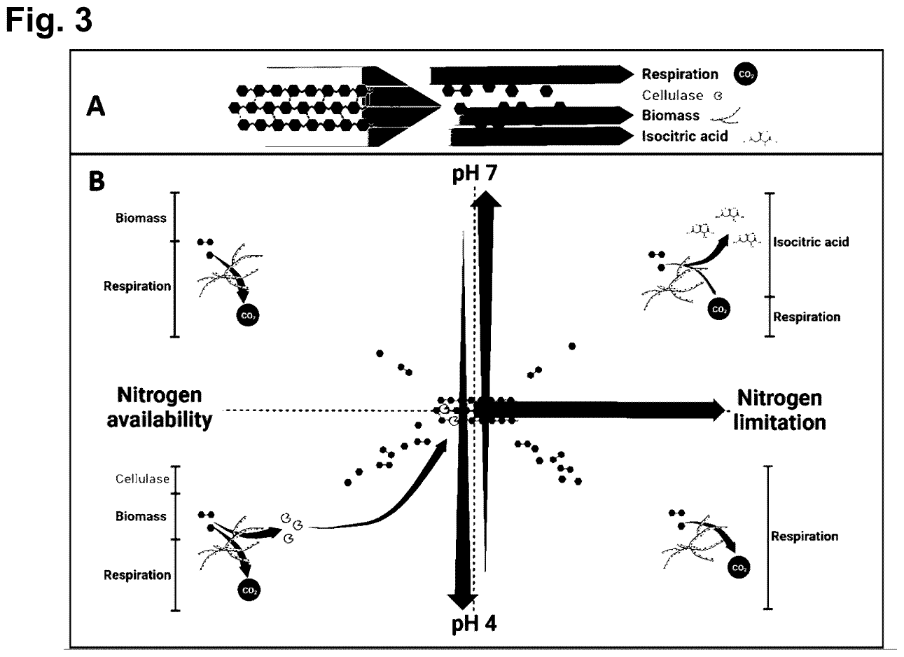

FIG. 3 shows the faith of cellulose utilization of P. verruculosum under different conditions. A) visualizes the major conversion pathways from cellulose. The rate limiting step is the hydrolysis of cellulose to soluble sugars which then are converted to either fungal biomass, CO₂ (respiration), cellulase enzymes or D-erythro-isocitric acid. B) shows the trend of the relative distribution of these four major products in the context of cultivation pH and nitrogen availability. A prerequisite for cellulose consumption is the formation of cellulases, which is only efficient below pH 6.5 and with nitrogen availability. Once cellulases have been produced the enzymes will continuously release soluble sugars from the cellulose, which can be converted to biomass, more cellulases enzymes, energy (respiration) or D-erythro-isocitric acid, depending on the conditions. The closer the pH is to the enzyme optimum of 4.2, the faster the sugar release rate and the faster the overall conversion rate (green arrows). At higher pH values, overall conversion rates are low and sugars will be either used for fungal biomass growth as long as nitrogen is available or for D-erythro-isocitric acid production, once nitrogen is limiting. In one embodiment, the fast and efficient conversion of cellulose to isocitric acid was therefore possible in a narrow range around pH 5.5 under nitrogen limitation. While the cellulose conversion rate can be faster below pH 5.5, isocitric acid formation was affected, limiting the overall space-time yield.

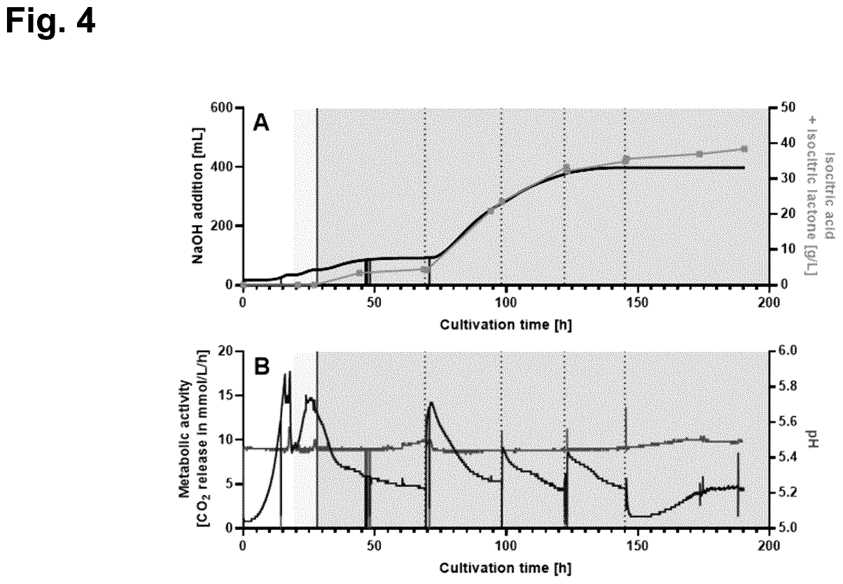

FIG. 4 shows the consolidated bioprocessing of cellulose to D-erythro-isocitric acid in a pH controlled stirred tank reactor. A) shows the D-erythro-isocitric acid production during the cultivation in comparison with the NaOH addition that was used for pH adjustment. Since isocitric acid is the only acid product, the base addition serves as a real-time signal for isocitric acid production. B) shows the metabolic activity of P. verruculosum during the fermentation measured via CO₂ release in the off-gas together with the cultivation pH. During the first 19 h of cultivation, the glucose and peptone in the medium were metabolized, leading to an exponential increase in the CO₂ release rate. After consumption of glucose and peptone, the metabolic activity dropped sharply, marking the transition to cellulase production phase (orange shaded). As cellulase concentration increases, the metabolic activity increases and the cellulose hydrolysis rate also increases. After 28 h the nitrogen sources are completely consumed and the fermentation becomes nitrogen limited (red vertical line). This leads to the D-erythro-isocitric acid production phase (blue shaded). The dotted black lines indicate feeding of cellulose powder (first 60 g/L, then 30 g/L, then 30 g/L, finally 60 g/L).

### EXAMPLES

### Introduction/Summary

Under the right conditions, the fungal strain Penicillium verruculosum is able to convert cellulosic substrates directly into D-erythro-isocitric acid with high titers and high productivity. The ability of P. verruculosum to produce isocitric acid as well as cellulolytic enzymes is already known. However, the direct production of isocitric acid from cellulose has not yet been demonstrated and only works under certain specific conditions. The invention therefore relates to a particular process that allows the effective conversion of cellulose to D-erythro-isocitric acid.

The production of isocitric acid from glucose with P. verruculosum only works at above pH 4, with a sharp drop in isocitric acid yield below pH 5 and a maximum yield above pH 5. In addition, the formation of isocitric acid is induced by a nitrogen limitation. The production of cellulolytic enzymes, on the other hand, requires a nitrogen source. In addition, a pH below 6.5 is required for the efficient production of cellulases. The activity optimum of the cellulases is also in the weakly acidic range at pH 4.2. The direct and efficient production of isocitric acid from cellulose is therefore only possible within a quite narrow process window in the pH range between 4 and 7, preferably at between 5 and 6. In addition, the initial amount of nitrogen in the medium must be such that, on the one hand, enough cellulases are formed and, on the other hand, the nitrogen is completely consumed in order to achieve nitrogen limitation.

In one embodiment, the method takes place in the following mineral medium:

| substance | concentration (g/L) |
|---|---|
| (NH₄)₂SO₄ | 2.5 |
| KH₂PO₄ | 2.6 |
| MgSO₄*7H₂O | 0.5 |
| CaCl₂*2 H₂O | 0.228 |
| NaCl | 0.05 |
| Citric acid | 0.45 |
| CoCl₂*6 H₂O | 0.006780251 |
| Fe₂(SO₄)₃ | 0.00573 |
| ZnSO₄*7H₂O | 0.04 |
| CuSO₄*5H₂O | 0.005113861 |
| H₃BO₃ | 0.002 |
| Peptone ex Casein | 2 |
| Glucose | 5 |
| α-Cellulose | 30 |

The pH value is ideally kept constant at 5.5 using NaOH, Ca(OH)₂ or CaCO₃. The process temperature can be preferably at between 3° and 37° C. While isocitric acid yield and productivity decrease slightly at higher temperatures, cellulose hydrolysis rate is increased at higher temperatures. The growth and cellulase production phase can last for example 28 hours, after which all nitrogen is consumed and isocitric acid production begins. After this phase, the process is run in fed batch mode and fresh, sterilized cellulose powder is fed in. The feeding can take place non-sterile with the fermenter lid open, the process has proven to be robust against contamination. The cellulose concentration in the process is limited by the rheology, above 120 g/L a classic submersed fermenter can no longer be stirred sufficiently. Therefore, 60 g/L cellulose was added as the first feeding, later portions of 30 g/L.

In this embodiment, at 30° C. and pH 5.5, cellulose turned into D-erythro isocitric acid at a maximum titer of 38.4 g/L, a maximum productivity of 0.7 g/L/h and a total yield of 0.24 g/g glucose equivalents based on the supplied amount of cellulose (including the cellulase production phase) or 0.29 g/g glucose equivalents (without cellulase production phase) (Table 1). The enantiomeric purity of the D-erythro-isocitric acid was >99.5%, and no other by-products such as citric acid were formed (HPLC).

With glucose as a substrate and using an excess of CaCO₃ as a pH regulator (fermentation pH>6), concentrations in excess of 100 g/L, a maximum productivity of 1.2 g/L/h and a maximum yield of 0.76 were obtained g/g reached. However, the average productivity in the glucose process dropped to 0.7 g/L/h. Therefore, P. verruculosum achieves comparable productivity on cellulose as on glucose. Although the performance achieved using cellulose is among the best values that have ever been achieved in consolidated bioprocesses, the process still shows great potential for optimization. The most important variables for further optimization seem the nitrogen concentration (more nitrogen->more biomass, more cellulases->higher hydrolysis rate->higher productivity), as well as pH value (optimal value between pH 5 and 6, compromise between maximum isocitric acid yield and maximum cellulase activity) and temperature (also on compromise between maximum isocitric acid yield and maximum cellulase activity). It is also not yet clear why the titer on cellulose does not continue to increase after reaching 38.4 g/L despite excess substrate. Without this limitation, the substrate would be digested more efficiently and yields well above 0.29 g/g would be achieved. The theoretically possible yield is 1.07 g/g.

### Materials and Methods

### Microorganisms

Penicillium verruculosum M28-10, kindly gifted by Dr. Gerhard Kerns (Saxon Institute for Applied Biotechnology. Leipzig. Germany), was propagated at 30° C. on medium containing 10 g/L malt extract (Difco: Becton, Dickinson and Company, USA), 40 mL/L carrot juice, 10 g/L wheat bran (Alnatura, Darmstadt, Germany), 10 g/L α-cellulose (Sigma-Aldrich, St. Louis, USA). 30 g/L agar (Difco; Becton. Dickinson and Company, USA). Spores were harvested from agar plates using 10 mL 0.01% (v/v) Tween 80 solution and washed twice with bi-distilled water. The spore concentration was determined in a Neubauer-Improved counting chamber (Superior Marienfeld, Lauda-Königshofen, Germany), adjusted to 10⁹ spores/mL and stored at 4° C. This 1000 concentrated stock was used for inoculation all experinents.

### Media and Cultivation

If not stated otherwise in the corresponding figure caption, all shake flask experiments were performed in 250 mL Erlenmeyer flasks with 25 mL filling volume at 30° C., 200 rpm and 50 mm shaking diameter. Cultures were inoculated with 10⁶ spores/mL.

The cultivation medium was based on a medium published by Pakula et al., which we modified into a universal mineral medium for compatibility with various organisms, and which we successfully employed for various processes including cellulase production, natural product formation as well as organic acid production by respective organisms [6, 19, 29, 30]. The version of the basal medium used here consisted of KH₂PO₄ 2.6 g/L, MgSO₄·7H₂O 0.5 g/L, CaCl₂·2H₂O 0.23 g/L, NaCl 0.05 g/L, peptone ex casein 2 g/L (N-Z-Amine® AS, Carl Roth, Karlsruhe, Germany). Tween 80 0.1% (v/v) and trace element solution 2.5 mL/L. Different buffers, carbon sources such as glucose and α-cellulose as well as different (NH₄)₂SO₄ concentrations were used as indicated in the respective figure captions of each experiment. The trace element solution (400×concentrated) had the following composition: citric acid 180 g/L, Fe₂(SO₄)₃ 2.29 g/L, ZnSO₄·7H₂O 16 g/L, CuSO₄ 2.05 g/L, MnSO₄·7H₂O 1.6 g/L, H₃BO₃ 0.8 g/L, CoCl₂·6H₂O 2.71 g/L.

The main solution without cellulose. NaCl and peptone was always prepared as a 2×concentrated stock solution that was set to the indicated starting pH with 5 M NaOH. The solution was filtered through a 0.2 μm tilter for sterilization. Before the experiment, the 2×concentrate was diluted to its original concentration by addition of sterile autoclaved 2×concentrate of the peptone-NaCl mixture.

### Investigation of Process Optima for Cellulase Production

For evaluating cellulase production optima, a previously published method based on online respirometry was applied, which measures the in situ cellulose consumption rate during growth in a medium containing 5 g/L glucose, 2 g/L peptone and 30 g/L cellulose as carbon sources [19, 31]. To achieve different pH profiles, the basal minimal medium was buffered with the following different buffer components, each at 100 mM concentration: MES (pH 5.4), PIPPS (pH 6.5) or CaCO₃ (pH 7.3). Cultivations were performed in a transfer rate online measurement device (Kuhner TOM) in 250 ml Erlenmeyer flasks with 25 ml filling volume at 30° C., 200 rpm and 50 mm shaking diameter. Cultures were inoculated with 10⁶ spores/mL. The in situ cellulose consumption performance in the different conditions was then evaluated based on the maximum O₂ consumption rate after exhaustion of glucose and peptone from the medium, which is indicated by a transient drop in O₂ consumption rate [19].

### Investigation of Process Optima for Cellulase Activity

Cell free supernatant of a P. verruculosum fermentation was diluted 5× in bi-distilled water and analyzed according to a downscaled filter paper assay adapted by Xiao modified for different pH values [32]. Instead of the 75 mM citrate buffer used in classical filter paper assay, the buffer was replaced by 75 mM phosphate-citrate buffer in order to set a broad range of pH values. Each column of the PCR plate was buffered to a different pH spanning a pH range of 2.6 to 7.4. The assay was performed in a 60 μL reaction volume in 96-well conical bottom PCR plates incubated for 1 h in a PCR thermocycler with temperature gradient function (Bio-Rad CFX connect). A temperature gradient was set to span a range between 3° and 50° C. across the rows of the 96-well plate to test the temperature vs. activity profile. The reducing sugars produced after 1 h incubation were quantified spectrophotometrically at 540 nm using the dinitrosalicylic acid method [32]. Finally, the measured values were normalized against the maximum value after blanking using a reaction containing bi-distilled water instead of diluted P. verruculosum supernatant.

### Investigation of Process Optima for Isocitric Acid Formation

To investigate the influence of pH on isocitric acid formation, two different batch cultivations with a starting pH of 6.7 using either 100 mM or 50 mM MES buffer were performed in order to achieve different dynamic pH profiles. The cultivation was performed in medium containing 1.25 g/L (NH₄)₂SO₄ and 60 g/L glucose as carbon source. The metabolic activity of the cultures was monitored using the TOM device to detect the onset of isocitric acid production as a change in RQ. After onset of isocitric acid production the cultures were sampled, to monitor the change in pH and the production of isocitric acid. After 65 h of cultivation, when the pH dropped and isocitric acid formation stopped, the culture was fed with an additional 40 g/L of glucose and the pH was adjusted to 6.5 using NaOH solution to investigate whether isocitric acid formation restarts after pH shift. The isocitric acid production yield was determined for the different intervals between measurements during the cultivation. Finally, the isocitric acid yield was plotted against the pH to investigate the relationship between pH and isocitric acid yield.

To investigate the influence of cultivation temperature on isocitric acid formation, cultivations were performed in medium with 1.25 g/L (NH₄)₂SO₄ and 60 g/L glucose, buffered with CaCO₃ (40 g/L), which has been shown to enable maximum isocitric acid yield in previous experiments. For each analyzed cultivation temperature (30°, 37° and 40° C.) a 100 mL culture was performed in a 1000 mL flask. After 3 days cultivation, the cultures were fed with 50 g/L glucose and split into triplicates of 25 mL cultures of which one replicate of each culture was shifted to either 30, 37 or 40° C., to investigate the effect of a decrease or increase in cultivation temperature in regard to the initial cultivation temperature. Samples were taken to calculate the isocitric acid productivity, glucose consumption rate and isocitric acid yields via linear regression.

### Investigation of Nitrogen Dependency for Isocitric Acid Formation

To investigate nitrogen dependency of isocitric acid formation, cultivations were performed in medium buffered with CaCO₃ (40 g/L) with 50 g/L glucose and (NH₄)₂SO₄ concentrations ranging from 1.25 to 5 g/L). The cultivations were performed in a TOM device for online respirometry to follow the metabolism and the timing of nitrogen limitation of the cultures. Samples were taken to measure glucose, isocitric acid and the remaining NH₄⁺ concentration expressed as g/L of (NH₄)₂SO₄ equivalents. NH₄⁺ was determined according to a modified version of the Berthelot reaction [33]. The method was scaled down to be realized in microtiter plates.

### Stirred Tank Fermentation of Cellulose to Isocitric Acid

The fermentation was performed in using a Biostat B-DCU fermenter (B.Braun International, Melsungen, Germany) equipped with one 6.4 cm diameter Rushton turbine in a 5 L vessel. The fermentation volume was 2.5 L. The fermenter was autoclaved with 75 g α-cellulose and 1.25 L of a 2×concentrate of the peptone-NaCl mixture. The other medium ingredients were supplied after autoclavation as 1.25 L of a sterile filtered 2×concentrate. The fermentation medium contained 2.5 g/L (NH₄)₂SO₄, with no buffer component as the pH was constantly regulated to 5.5 using 10% NaOH. Tween 80 was omitted to prevent excessive foaming. The fermenter was inoculated with 250 mL (10%) of 5 day old pre-culture grown in the same medium but including Tween 80 and 100 mM PIPPS buffer with starting pH adjusted to pH 5.4 with NaOH. The stirring rate was 600 rpm the first 24 h, then increased to 700 rpm from 24-70 h and finally to 800 until end of fermentation. The gassing rate was constant at 1 VVM (2.5 L/min). The temperature was regulated to 30° C. Cellulose was fed as portions of dry autoclaved powder of 150 g, 75 g, 75 g, 150 g after 69 h, 98 h, 122 h and 148 h, respectively. This was realized by opening the complete lid of the fermenter, the feeding was therefore only semi-sterile.

### HPLC Analytics

Erythro-isocitric acid and its lactone, threo-isocitric acid and its lactone, and citric acid were quantified via HPLC analysis (X-LC, JASCO International Co. Tokyo, Japan) at 25° C. using the following setup: Column: TSKgel ODS-100V, 250×4.6 mm, 5 μm (Tosoh Bioscience, Tokyo. Japan); UV/VIS detector (UV-4070, JASCO International Co. Tokyo, Japan) at 210 nm and refractory index detector (RI-4035. JASCO International Co. Tokyo, Japan); mobile phase: 0.1% H₃PO₄ (v/v); flow rate: 0.5 mL/min. Thereby, the UV-detector was used for quantification. The samples were diluted 1/10 in 0.1 g/L fumaric acid solution as internal standard to compensate small deviations in injection volume.

Glucose was quantified via HPLC analysis (Dionex HPLC UltiMate 3000. Thermo Scientific. Waltham. USA) at 65° C. using the following setup: Column: AMINEX Ion Exclusion HPX-87H, 300-7.8 mm (Bio-Rad Laboratories GmbH, Munich, Germany); detectors: Dionex™ Ultimate 3000 UV/VIS detector (Thermo Scientific, Waltham, USA) at 210 nm and RI-101 refractory index detector (Shodex, Munich. Germany); mobile phase: 5 mM sulfuric acid; flow rate: 0.7 mL/min. Thereby, the RI-detector was used for quantification of glucose.

To dissolve potentially precipitated calcium isocitrate in cultivations using CaCO₃ as buffer, the broth was diluted 3 × with 1 M HCl. After centrifugation of the fermentation samples (16.900 g; 10 min; 4° C.) and a second centrifugation step of the resulting supernatant (3,000 g: 10 min), the supernatant was analyzed by HPLC.

### Different Optima for the Different Subprocesses as a Challenges of Consolidated Bioprocessing

In a consolidated bioprocess, the first bottleneck is the hydrolysis of cellulose into soluble sugars. During the cultivation of cellulolytic organisms on cellulose, the hydrolysis rate of cellulose typically doesn't exceed sugar consumption rate of the organism, hence sugars don't accumulate but are consumed instantly. This is of benefit for the process since product inhibition of cellulases by sugar mono- and oligomers is completely avoided. However, as a result, the rate of cellulose hydrolysis will ultimately limit the formation rate of isocitric acid. Thereafter, the isocitric acid formation will mainly depend on the yield from the released sugars. Hence, the goal is to find conditions that maximize the cellulose hydrolysis rate while ensuring the highest possible isocitric acid t yield.

The net cellulose hydrolysis rate depends primarily on the cellulose concentration, the cellulose digestibility, the cellulase concentration and the cellulase activity profile [19]. Conditions therefore should be chosen to produce sufficient cellulase enzymes but also stay close to the cellulase activity optimum.

Therefore, the inventors reanalyzed the cellulase production profile of P. verruculosum according to a published method based on online respirometry under different conditions [19]. This method consists of growing the fungus on a medium containing 5 g/L glucose for initial growth and 30 g/L of cellulose. The glucose consumption precedes the cellulose consumption and can be differentiated from cellulose consumption by a distinct metabolic peak. The red trace in FIG. 2A shows these distinct peaks for the condition optimized for highest cellulase production (buffered to pH 5.4 with PIPPS). In contrast, cultures buffered to pH 6.5 with MES only reached very low cellulose consumption rates after glucose exhaustion. Cultures that were buffered with CaCO₃ to keep the pH above 6.5 were completely unable to consume cellulose and could only consume glucose. It was concluded that efficient cellulase production needs pH values below pH 6.5.

Regarding temperature, similar results have been obtained with temperatures ranging between 3° and 37° C. [19]. Moreover, the activity optimum of the cellulase enzymes was analyzed as shown in FIG. 2B. Consistent with the cellulase production profile, the pH optimum of the enzymes was in the acidic range at a pH of 4.2, with a steep increase in activity up to a temperature of 50° C. These values are in good agreement with the pH optimum determined by other authors [28]. Lastly, the process optimum for the isocitric acid formation was investigated by performing different cultivations of P. verruculosum using glucose as carbon source. When analyzing different pH profiles using different buffer concentrations, it became obvious that isocitric acid formation is only observed for pH values above 4 with a sharp drop in isocitric acid yield below pH 5.5 (FIG. 2C). When analyzing different temperatures, both isocitric acid yield and productivity were in tendency slightly lower at 37° C. (0.68 g/g and 0.68 g/L/h) compared to 30° C. (0.75 g/g and 0.82 g/L/h) (FIG. 2D).

Because of the opposing pH activity profiles, cellulose conversion into isocitric acid is only possible in a narrow process window around pH 5, in particular between 4.8 to 5.8. Another opposition in process requirements lies in the availability of nitrogen. When testing cultivations with different nitrogen concentrations, we found that isocitric acid formation only starts after nitrogen limitation. On the other hand, the formation of microbial biomass and cellulases are directly dependent on the availability of nitrogen. Therefore, isocitric acid production cannot proceed simultaneously to cellulase production but must be executed in sequence to cellulase production. The starting concentration of nitrogen will therefore affect the final amount of microbial biomass and cellulases produced as well as the time until nitrogen limitation is reached. The nitrogen concentration in the medium and the pH are therefore the most influential factors on the system and determine the faith of carbon utilization in the culture as depicted in FIG. 3. While a high pH in combination with nitrogen limitation is a requirement for efficient isocitric acid formation, cellulase activity and thus cellulose conversion will be negligible and no cellulases can be produced under these conditions. In contrast, a low pH will ensure high cellulase activity and thus cellulose conversion efficiency but prevent isocitric acid formation regardless of nitrogen availability.

### Direct Conversion of Cellulose to D-Erythro-Isocitric Acid

Under conditions optimized for cellulase production, the inventors previously demonstrated using respirometry that P. verruculosum can reach in-situ cellulose metabolization rates of 0.9 g/L/h at low cellulose concentration (30 g/L) and even 1.7 g/L/h at high cellulose concentrations (120 g/L) [19]. For D-erythro-isocitric acid formation, glucose consumption rates of 1.4 to 2.6 were measured depending on the starting concentration of nitrogen, which resulted in isocitric acid production rates between 0.7 to 1.2 g/L/h, respectively. However, the high initial glucose consumption rates observed with high nitrogen supplementation could be only sustained for limited time and later dropped to a value of 1.4 g/U/h together with a drop in D-erythro-isocitric acid productivity to 0.7 g/L/h. Hence, in theory, P. verruculosum has the cellulolytic capacity to produce D-erythro-isocitric acid from cellulose at a similar rate as from glucose. To test whether this can be also achieved experimentally, we tested the CBP in a pH controlled stirred tank fermenter at pH 5.5, which should lead to high cellulase formation with only minor impact on D-erythro-isocitric acid yield according to FIG. 2C. In contrast to a standard cellulase fermentation setup, only a low quantity of 2.5 g/L (NH₄)₂SO₄ was supplied to reach nitrogen limitation after growth and cellulase production.

The fermentation was performed in a 5 L vessel with 2.5 L medium inoculated with 250 mL of 5 day old pre-culture. The stirring rate was 600 rpm from 0-24 h, 700 rpm from 24-70 h and 800 rpm from 70 until end of fermentation. The gassing rate was 2.5 L/min, temperature 30° C., pH was regulated to 5.5 using 10% NaOH.

As can be seen in FIG. 4B, the metabolic activity profile that typically indicated high cellulase formation was achieved in the first 70 h of cultivation [19]. A peak CO₂ release rate of 15 mmol/Uh was reached after 25 h of cultivation, close to the value achieved in shake flasks under optimized conditions (FIG. 2A). As intended, after 28 h a nitrogen limitation kicked in, which induced D-erythro-isocitric acid production. Between 27 h and 44 h, 3.5 g/L isocitric acid were produced, corresponding to a slow production rate of 0.2 g/L/h. As the sugar release rate is proportional to the cellulose concentration, 60 g/L of fresh cellulose were fed after 70 h to boost production. It can be seen that the isocitric acid production rate instantly increased and a concentration of 23.5 g/L D-erythro-isocitric acid was reached until the next feeding interval, reaching a peak productivity of 0.7 g/L/h. This value is well comparable with glucose-based fermentations. In order to keep the production rate high, three further cellulose feedings were performed. Despite the feeding, the isocitric acid formation rate declined continuously and came to a stop at a concentration of 38.4 g/L, even though the cellulose concentration was high. It is not known yet, what inhibited the production at later stages of the fermentation. However, in glucose-based cultivations, concentrations of 100 g/L were achieved, highlighting still a big potential for further process optimization.

Because D-erythro-isocitric acid might be an interesting new chemical building block for chiral synthesis, the enantiomer purity of the cellulose based isocitric acid was analyzed. In total, 38.4 g/L isocitric acids were produced including isocitric lactones. Of these, 99.5% were D-erythro-isocitric acid. This distribution was similar to the D-erythro-isocitric acid based on glucose. The different species of isocitric acid are shown in table 2. No other major side products were detected as can be seen from the refractive HPLC spectra.

TABLE 2
Overview of different isocitric acid species produced
after fed-batch cultivation with cellulose or glucose.

| | cellulose | glucose |
|---|---|---|
| D-erythro-Isocitric acid [g/L] | 24.8 | 84.7 |
| D-threo-Isocitric acid [g/L] | 0.18 | 0.17 |
| D-erythro-Isocitric acid lactone [g/L] | 13.6 | 16.4 |
| D-threo-Isocitric acid lactone [g/L] | 0 | 0 |
| Total isocitric acids [g/L] | 38.4 | 101.3 |
| D-Erythro-isocitric acid [%] | 99.5 | 99.8 |

### REFERENCES

1. Alper, H. and G. Stephanopoulos, Engineering for biofuels: exploiting innate microbial capacity or importing biosynthetic potential? Nature Reviews Microbiology, 2009. 7(10): p. 715-723.
2. Li, T., et al., Unique genetic cassettes in a <i>Thermoanaerobacterium</i> contribute to simultaneous conversion of cellulose and monosugars into butanol. Science Advances, 2018. 4(3): p. e1701475.
3. Jin, M., et al., Consolidated bioprocessing (CBP) of AFEX™-pretreated corn stover for ethanol production using Clostridium phytofermentans at a high solids loading. Biotechnol Bioeng, 2012. 109(8): p. 1929-36.
4. Zuroff, T. R., S. B. Xiques, and W. R. Curtis, Consortia-mediated bioprocessing of cellulose to ethanol with a symbiotic Clostridium phytofermentans/yeast co-culture. Biotechnology for Biofuels, 2013. 6(1): p. 59.
5. Li, Q., J. A. Siles, and I. P. Thompson, Succinic acid production from orange peel and wheat straw by batch fermentations of Fibrobacter succinogenes S85. Appl Microbiol Biotechnol, 2010. 88(3): p. 671-8.
6. Schlembach, I., et al., Consolidated bioprocessing of cellulose to itaconic acid by a co-culture of Trichoderma reesei and Ustilago maydis. Biotechnology for Biofuels, 2020. 13(1): p. 207.
7. Scholz, S. A., et al., Production of cellulosic organic acids via synthetic fungal consortia. Biotechnol Bioeng, 2018. 115(4): p. 1096-1100.
8. Chi, X., et al., Hyper-production of butyric acid from delignified rice straw by a novel consolidated bioprocess. Bioresource Technology, 2018. 254: p. 115-120.
9. Shahab, R. L., et al., Consolidated bioprocessing of lignocellulosic biomass to lactic acid by a synthetic fungal-bacterial consortium. Biotechnology and Bioengineering, 2018. 115(5): p. 1207-1215.
10. Brethauer, S. and M. H. Studer, Consolidated bioprocessing of lignocellulose by a microbial consortium. Energy & Environmental Science, 2014. 7(4): p. 1446-1453.
11. Minty, J. J., et al., Design and characterization of synthetic fungal-bacterial consortia for direct production of isobutanol from cellulosic biomass. Proceedings of the National Academy of Sciences, 2013. 110(36): p. 14592-14597.
12. Wen, Z., et al., Enhanced solvent production by metabolic engineering of a twin-clostridial consortium. Metabolic Engineering, 2017. 39: p. 38-48.
13. Lynd, L. R., et al., Consolidated bioprocessing of cellulosic biomass: an update. Curr Opin Biotechnol, 2005. 16(5): p. 577-83.
14. Schlembach, I., et al., Measurement Techniques to Resolve and Control Population Dynamics of Mixed-Culture Processes. Trends Biotechnol, 2021. 39(10): p. 1093-1109.
15. Rarbach, M. and Y. Söltl, Cellulosic Ethanol from Agricultural Residues. MTZ worldwide, 2013. 74(4): p. 4-8.
16. Hortsch, R. and P. Corvo, The Biorefinery Concept: Producing Cellulosic Ethanol from Agricultural Residues. Chemie Ingenieur Technik, 2020. 92(11): p. 1803-1809.
17. Peyronel, B., I germi atmosferici dei funghi con micelio: contributo all'aerospermologia degli eumiceti: sunto della dissertazione per la laurea in scienze naturali di Beniamino Peyronel. 1913: Tip. Gallina.
18. Solov'eva, I. V., et al., The selection and properties of Penicillium verruculosum mutants with enhanced production of cellulases and xylanases. Microbiology, 2005. 74(2): p. 141-146.
19. Antonov, E., et al., Process relevant screening of cellulolytic organisms for consolidated bioprocessing. Biotechnology for Biofuels, 2017. 10(1): p. 106.
20. Beppu, T., S. Abe, and K.-i. Sakaguchi, Accumulation of Isocitric Acid by a Penicillium Strain. Bulletin of the Agricultural Chemical Society of Japan, 1957. 21(4): p. 263-264.
21. Kinichiro, S. and B. Teruhiko, Method of producing allo-isocitric acid by fermentation. 1960, Google Patents.
22. RÅnby, M., et al., Isocitrate as Calcium Ion Activity Buffer in Coagulation Assays. Clinical Chemistry, 1999. 45(8): p. 1176-1180.
23. GmbH, C. What if scientists made a further substantial contribution to the fountain of youth? 2021.
24. GmbH, C. The hidden Gem Isocitric Acid—Isocitrate. [cited 2022 23.11.2022]; Available from: https://www.chiroblock.com/isocitric-acid-buy-chiroblock/.
25. Moore, G. L., et al., Practical Synthesis of the Bicyclic Darunavir Side Chain: (3R,3aS,6aR)-Hexahydrofuro[2,3-b]furan-3-ol from Monopotassium Isocitrate. Org Process Res Dev, 2017. 21(1): p. 98-106.
26. Werpy, T. and G. Petersen, Top value added chemicals from biomass: volume I—results of screening for potential candidates from sugars and synthesis gas. 2004, National Renewable Energy Lab., Golden, CO (US).
27. Berg, R. G., Preparation of citraconic and itaconic acids. 1972, Google Patents.
28. Morozova, V. V., et al., Cellulases of Penicillium verruculosum. Biotechnol J, 2010. 5(8): p. 871-80.
29. Pakula, T. M., et al., The effect of specific growth rate on protein synthesis and secretion in the filamentous fungus Trichoderma reesei. Microbiology, 2005. 151(1): p. 135-143.
30. Palacio-Barrera, A. M., et al., Reliable online measurement of population dynamics for filamentous co-cultures. Microbial Biotechnology, 2022. 15(11): p. 2773-2785.
31. Antonov, E., et al., Efficient evaluation of cellulose digestibility by Trichoderma reesei Rut-C30 cultures in online monitored shake flasks. Microbial Cell Factories, 2016. 15(1): p. 164.
32. Xiao, Z., R. Storms, and A. Tsang, Microplate-based filter paper assay to measure total cellulase activity. Biotechnology and Bioengineering, 2004. 88(7): p. 832-837.
33. Krom, M. D., Spectrophotometric determination of ammonia: a study of a modified Berthelot reaction using salicylate and dichloroisocyanurate. The Analyst, 1980. 105(1249): p. 305-316.

1. A method for a one-step bioproduction of isocitric acid from a complex carbohydrate substrate in a suitable fungus, the method comprising:
cultivating a cell culture comprising a suitable fungus, the fungus being of the genus that comprises one of Penicillium, Talaromyces or Aspergillus, in a culturing vessel in a suitable medium comprising a complex carbohydrate at a temperature of between 25° and 40° C., under nitrogen limitation, and at a pH of the cell culture at between 4 to 7.

2. A method according to claim 1, wherein the isocitric acid is D-erythro-isocitric acid ((2S,3S)-isocitric acid).

3. A method according to claim 1, wherein the Penicillium is selected from a strain that is at least one of non-genetically modified, native, or wildtype cellulolytic strain of the Penicillium.

4. A method according to claim 1, wherein the temperature is at 30° C. to 37° C.

5. A method according to claim 1, wherein the pH of the cell culture is at between 5.3 to 5.7.

6. A method according to claim 1, wherein the complex carbohydrate is selected from resides that are one of agricultural or agroforestry residues and that comprise one or more of starch, pectin, and cellulose.

7. A method according to any one of claims 1 to 6, claim 1, wherein culturing the cultivation comprises stirring at a rate based on a power input of between 0.5 to 10 kW m3 of the cell culture.

8. A method according to any one of claims 1 to 7, claim 1, wherein one of: no nitrogen is added to the cell culture, or the nitrogen is limited to less than 0.5 g/L of the cell culture.

9. A method according to claim 1, wherein a gassing rate of the cell culture is at between 0.2 and 2 volumes of air sparged per unit volume of growth medium per minute (VVM).

10. A method according to any one of claims 1 to 9, claim 1, wherein said culture is performed the cell culture is a fed-batch culture, for example comprising culture and the cultivation comprises feeding of the complex carbohydrate in order to maintain the level of substrate complex carbohydrate in the cell culture at above about 60 g/L.

11. A method according to any one of claims 1 to 10, claim 7, further comprising the step of isolating the isocitric acid as produced from the cell culture, comprising, for example comprising in-situ removal of the isocitric acid.

12. A method according to wherein 30-45 g/L are produced and the isocitric acid comprises more than 99% D-erythro-isocitric acid.

13. A kit, comprising materials for performing the method according to any one of claims 1 to 12, such as, for example, claim 1, further comprising a culture of a suitable fungus, in particular the fungus being of the genus that comprises one of Penicillium, Talaromyces or Aspergillus, the kit further comprising a suitable culture medium comprising lacking nitrogen and having a pH at between 4 to 7, preferably between 4.8 to 5.8, more preferably at 5.5. 7.

14. (canceled)

15. A method according to claim 3, wherein the strain of the Penicillium comprises one of Penicillium verruculosum M28-10 or Penicillium verruculosum M28-9.

16. A method according to claim 5, wherein the pH of the cell culture is at 5.5.

17. A method according to claim 6, wherein the complex carbohydrate is derived from straw.

18. A method according to claim 9, the gassing rate of the cell culture is at 1 VVM.

19. A method according to claim 12, the isocitric acid comprises 99.5% D-erythro-isocitric acid.

20. A kit according to claim 13, wherein the pH is at between 4.8 to 5.8.

21. A kit according to claim 20, wherein the pH is at 5.5.
